(19) United States
(12) Patent Application Publication
(10) Pub. No.: US 2026/0097541 A1
(43) Pub. Date: Apr. 9, 2026

(54) METHOD AND SYSTEM FOR DETECTING AND REGULATING WORKABILITY OF CONCRETE IN CONCRETE MIXER TRUCK

(71) Applicant: Huazhong University Of Science And Technology, Wuhan City (CN)

(72) Inventors: Yongbing XU, Wuhan City (CN); Yongmo XU, Wuhan City (CN)

(73) Assignee: Huazhong University Of Science And Technology, Wuhan City, HB (CN)

(21) Appl. No.: 19/415,575

(22) Filed: Dec. 10, 2025

(30) Foreign Application Priority Data
Dec. 19, 2024 (CN) 202411874065.9

(51) Int. Cl. B28C 5/42; G01N 11/02; G01N 33/38

(52) U.S. Cl. CPC B28C 5/422; G01N 11/02; G01N 33/383

(57) ABSTRACT

A method and system for detecting and regulating workability of concrete in a concrete mixer truck are disclosed in the present disclosure. A rheological shear device and a temperature testing device are installed inside a drum of the concrete mixer truck. Rotation of the drum is used to drive a shear rod to shear in the concrete, which can directly test rheological properties of the concrete, and obtain work properties through a relationship between rheological properties and workability. A test is made before a concrete mixer truck leaves a factory. Rheological properties of the concrete are continuously measured during transportation, and change degree and change trend of the rheological properties are evaluated. An evaluation result is compared with a limiting value. When the limiting value is reached or exceeded, an alarm is immediately triggered and regulation of supplement admixtures is automatically performed.

### CROSS-REFERENCE TO RELATED APPLICATION

This application claims priority to Chinese Patent Application No. 202411874065.9, filed on Dec. 19, 2024, which is hereby incorporated by reference in its entirety.

### TECHNICAL FIELD

The present disclosure belongs to the technical field of detecting and regulating workability of fresh concrete, and more specifically, relates to method and system for detecting and regulating workability of concrete in concrete mixer truck.

### BACKGROUND ART

At present, quality control of ready-mix concrete primarily relies on laboratory mix design and factory production control. However, during transportation to the construction site, changes in the concrete's workability inside the concrete mixer truck are usually not actively and effectively managed. This is because the total duration from the plant to delivery is highly variable and influenced by factors such as transport distance, traffic congestion, unpredictable events like accidents and the waiting time at the construction site.

Changes in ready-mix concrete's workability result from cement hydration, the process is begun upon contact with water and leads to a continuous reduction in the concrete's moisture content. Owing to the aforementioned factors, changes in workability during the delivery process from the factory to the construction site can be unpredictable. If the workability deviates from the specified tolerance, the concrete is likely to be rejected by the contractor, resulting in waste. Even if accepted and used, such deviations can compromise the quality and safety of the concrete structure. Therefore, it is essential to monitor and adjust the concrete workability during this period.

To monitor and adjust concrete workability during delivery, indirect testing methods have been proposed. These methods can be divided into two categories: (1) a method that measures the change in the driving force of the drum of the concrete mixer truck during the rotation of the drum, and (2) a method that analyzes the concrete flow behavior inside the drum through visual imaging. However, these methods suffer from limitations in sensitivity and accuracy, especially in tracking time-dependent changes in workability for certain types of concrete, such as highly flowable mixtures. Consequently, the measurements cannot be reliably used as a guide for adjusting workability and mix proportions.

The workability monitoring and adjustment during the delivery process serve not only to ensure that concrete meets project specifications, but also to provide rheological data for optimizing the concrete mix design.

### SUMMARY

The present disclosure provides method and system for directly detecting and regulating workability of concrete in concrete mixer truck, to address deficiencies in existing technologies by improving the accuracy and real-time performance of workability measurements.

In order to achieve aforementioned objectives, according to the first aspect of the present disclosure, a method for detecting workability of concrete in concrete mixer truck is provided. The method includes following steps:

A rheological shear device, mounted through the drum wall from the exterior to the interior with its rheological shear rod projecting into the concrete, directly converts the resistance on the rheological shear rod into a pressure signal. This rheological shear device includes the rheological shear rod and a pressure sensor. Based on the lever principle, the pressure sensor transforms the mechanical resistance experienced by the rheological shear rod into a proportional pressure.

Based on the pressure signals and the corresponding rotation speeds of the drum, a linear relationship between the rotation speed and the resistance can be established. The intercept of the line of the linear relationship corresponds to the yield value, and the slope of the line of the linear relationship corresponds to the plastic viscosity. The time-varying rheological properties at different stages of delivery (e.g., beginning, middle, and end) can be used to optimize the concrete mix design.

Continuous monitoring of concrete workability during transportation is achieved by measuring the apparent viscosity at the drum's rotation speed. These real-time changes in apparent viscosity directly indicate in concrete workability.

The apparent viscosity of the concrete is calculated at the rotation speed of the drum at preset time intervals, and its trend over time is tracked.

When the apparent viscosity deviates beyond preset thresholds, indicating decreased workability, or when its developmental trend diverges from database benchmarks, the system will trigger automatic admixture supplement to ensure that the values remain within defined control limits in a real time.

In some embodiments, the method further includes: measuring concrete temperature and temperature of the drum wall during the transportation. The effect of ambient temperature on concrete temperature and workability can be related and used for prediction of the temperature effect and the use of supplemental admixture to adjust workability.

According to the second aspect of the present disclosure, a method for regulating workability of concrete in a concrete mixer truck is provided. The method includes: pre-establishing a database of a time-varying relationship of apparent viscosity of concrete with different categories, strength grades, and workability requirements under different transportation conditions; and pre-establishing a database of an effect of admixtures used for adjusting workability of different concretes during transportation.

In some embodiments, if the workability (as reflected by apparent viscosity measurement) decreases and the apparent viscosity reaches a preset threshold, the method for regulating workability further includes:

Based on the pressure data (measured at relevant drum rotation speeds) and the change in concrete temperature, an optimization algorithm is used to calculate the deviations of apparent viscosity, the yield value, and the plastic viscosity to automatically decide whether admixtures need to be supplemented.

In some embodiments, a variety of the admixtures which are equipped with the concrete mixer truck, an amount of each supplement, frequency of supplement and the type of the admixtures are automatically optimized and decided using an optimization algorithm according to a set target of the workability of concrete when delivery.

In some embodiments, the method further includes: monitoring time and an actual route of a concrete mixer truck during transportation and time waiting for delivery on the construction site, to assist in regulation of the workability of concrete.

According to the third aspect of the present disclosure, a system for detecting workability of concrete in concrete mixer truck is provided. The system includes:
a rheological shear device mounted through the wall of the drum of a concrete mixer truck. The rheological shear device includes a rheological shear rod that inserts into the concrete and a pressure sensor. Based on the lever principle, the rheological shear device converts the resistance experienced by the rheological shear rod during rotation into a pressure signal read by the pressure sensor.

The rheological parameters are calculated based on the relationship between the drum's rotation speed and the pressure signal, which is converted from the resistance experienced by the rheological shear rod. A linear relationship is observed at lower rotation speeds, where the intercept of the line of the linear relationship represents the yield value and the slope of the line of the linear relationship represents the plastic viscosity. The rheological parameters may be changed with time during delivery, reflecting the change of concrete workability and mix characteristics.

Real-time detection of concrete workability variation relies primarily on monitoring changes in apparent viscosity. The apparent viscosity is calculated from a pressure signal, which is converted by the resistance experienced by the rheological shear rod at the drum's rotation speed during transportation.

In some embodiments, the system further includes: a temperature sensor;

The temperature sensors are configured to measure three temperatures relevant to the cement hydration process: the concrete temperature inside the drum, the ambient temperature outside the drum, and the drum wall temperature. These real-time measurements enable the evaluation of temperature's effect on cement hydration acceleration and concrete workability. The assessment will determine if a preventive supplement of admixtures should be automatically triggered.

According to the fourth aspect of the present disclosure, a system for regulating workability of concrete in concrete mixer truck is provided. The system includes:
a database construction module, configured to pre-construct a database of a time-varying relationship of apparent viscosity of concrete with different categories, strength grades, and workability requirements under different transportation conditions;
a workability detection system, configured to detect the workability of concrete at different time during transportation in real time until the concrete mixer truck reaches a delivery location; where the workability detection system is the system for detecting workability of concrete in concrete mixer truck according to the third aspect;
a calculation module, configured to calculate a corresponding apparent viscosity at a time period during transportation based on real-time detected yield value and plastic viscosity; and compare the apparent viscosity with an apparent viscosity in the database to obtain a trend of apparent viscosity difference over time during the time period; and
a device for automatically supplementing spray admixtures, configured to automatically supplement admixtures when the trend of apparent viscosity difference over time during the time period leads to a decrease in workability and when the trend reaches a preset warning value, to regulate the workability of concrete in real time.

Overall, the above technical solutions conceived by the present disclosure can achieve the following beneficial effects.
(1) In the method for detecting workability of concrete in concrete mixer truck described in the present disclosure, a rheological shear device arranged inside a drum of a concrete mixer truck is used to directly convert rheological shear resistance experienced by a rheological shear rod into a pressure signal through a law of lever. The rheological shear rod is inserted into the concrete in the drum and shears the concrete during rotation of drum. Based on the pressure signal obtained by the rheological shear rod during rotation of the drum, a linear relationship between a rotation speed of the drum and a resistance experienced by the rheological shear rod is obtained. The rotation speed of the drum is proportional to the shear rate, and the resistance of the rheological shear rod is proportional to the shear stress. At a lower speed range, the rheological properties of concrete generally exhibit a Bingham behavior, that is, the shear speed and the shear resistance have a linear relationship. Therefore, the obtained relationship can characterize the rheological properties of the concrete in the drum. The intercept represents the yield value, and the slope of the straight line represents the plastic viscosity. The values of rheological parameters reflect the characteristics of the concrete's composition and the properties of fresh concrete, such as the paste volume ratio, flocculated structure, and aggregate gradation. These values can, therefore, be used to optimize the concrete proportioning design.
(2) As the rheological shear rod continuously shears the concrete at the drum's low rotational speed during transportation and on-site waiting, the system provides continuous measurements of the apparent viscosity. The data collected during shear-flowing of concrete inside the rotated drum is the direct test data on concrete and has advantages of sensitive, high accuracy, real-time performance, and independence from the different shapes and size of drum of the concrete mixer truck. The sensitivity to small changes in resistance, which can be caused by a reduction in moisture content or the segregation of coarse aggregates, provides valuable data for optimizing the concrete composition and supplementing admixtures. Therefore, the present disclosure not only enables the direct detection and adjustment of the workability of fresh concrete, but also provides a means for optimizing concrete proportioning.
(3) Furthermore, the present disclosure accounts for the effect of temperature on cement hydration in concrete. An increase in temperature can accelerate hydration, leading to reduced moisture and increased viscosity. Instead of directly measuring moisture, the disclosed method monitors in real-time the temperature difference between the concrete and the ambient during delivery. By establishing relationships between the yield value, plastic viscosity, apparent viscosity, and this temperature difference over time, the method improves the accuracy of predicting subsequent workability changes and enhances the precision of pre-adjusting concrete workability with supplementary admixtures.
(4) Preferably, the rheological shear rod is a sheet-like rod, and can be developed into a product series in length and cross-sectional shape. Different rheological shear rods can be selected according to the shear resistance of concretes with different workability from normal to highly flowability. This enhances the measurement sensitivity and accuracy.
(5) Furthermore, based on the method for detecting concrete workability in a concrete mixer truck as provided in the present disclosure, a corresponding regulation method is also established. This method automatically triggers the supplement of admixtures to ensure that the workability meets requirements. The decision is made when the apparent viscosity indicates a trend of increasing viscosity over time at a given ambient temperature and exceeds a preset warning value, based on real-time detection and a pre-constructed database of preset values.
(6) Furthermore, with the support of the database, and based on prediction of the apparent viscosity trends (especially when hydration temperature and time are major factors), an optimization algorithm intelligently determines whether admixtures are needed, what type is required, in what quantity, and how frequently they should be automatically added to pre-adjust the hydration process. This method provides more accurate and effective regulation of rheological properties than post-change adjustment, ensuring that workability requirements are consistently met.
(7) Furthermore, the present disclosure also includes: real-time monitoring the delivery time and actual route of the concrete mixer truck. This capability allows the system to address potential unexpected overtime or route deviations during transportation and on-site waiting, enabling timely intervention for quality management.

In summary, the method of the present disclosure enables direct assessment and regulation of the workability of ready-mix concrete in a concrete mixer truck drum during transportation and on-site waiting. The system, centered on a rheological shear device installed on the drum wall, directly and continuously collects rheological data from the fresh concrete while simultaneously monitoring key influencing factors such as hydration time and temperature. Based on this data, intelligent regulation is implemented to effectively and timely adjust the rheological properties, ensuring that workability requirements are met. Furthermore, the present disclosure provides a tool for owners, engineering supervisors, and regulatory agencies to monitor the concrete quality management of producers and suppliers online.

### BRIEF DESCRIPTION OF THE DRAWINGS

FIG. 1 is a schematic diagram of the method for detecting workability of concrete in concrete mixer truck according to embodiments of the present disclosure.

FIG. 2 is a schematic diagram of changes of rod torque over time according to embodiments of the present disclosure.

FIG. 3 is a schematic diagram of the relationship between rod torque and rotation speed under different cement conditions according to embodiments of the present disclosure.

### DETAILED DESCRIPTION OF THE EMBODIMENTS

In order to further clarify the purpose, technical solution, and advantages of the present disclosure, a detailed explanation of the present disclosure will be further provided in conjunction with the accompanying drawings and embodiments. It should be understood that the specific embodiments described herein are only for explaining the present disclosure and are not intended to limit the present disclosure. In addition, the technical features involved in the various embodiments of the present disclosure described below can be combined with each other as long as they do not conflict with each other.

### Embodiment 1

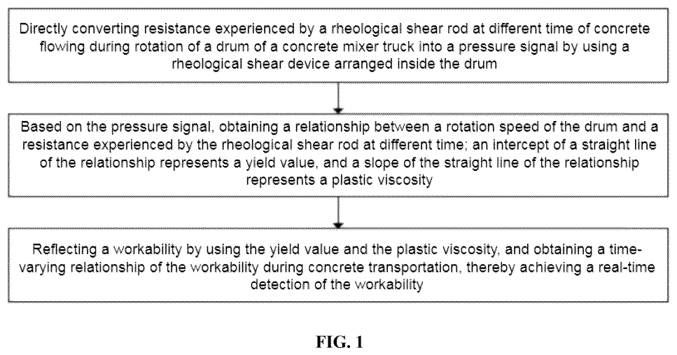

As shown in FIG. 1, a method for detecting workability of concrete in concrete mixer truck based on rheological performance test is provided in an embodiment of the present disclosure. The method mainly includes following steps S1 to S2.

In S1, a rheological shear device arranged inside a drum of a concrete mixer truck is used to directly convert resistance experienced by the rheological shear device when concrete flows at different time during rotation of the drum of the concrete mixer truck into a pressure signal. The rheological shear device designed in the embodiment of the present disclosure includes a rheological shear rod designed by a law of lever and a pressure sensor. During rotating of the drum of the concrete mixer truck, the rheological shear rod inserted into the concrete inside the drum undergoes resistance of concrete flow, and the resistance is converted into a pressure experienced by the pressure sensor through the law of lever. Through the rheological shear device, the resistance experienced by concrete flow at the rotation speed of the drum can be directly converted. through a fulcrum, into pressure on the pressure sensor in the drum. This pressure data is used to characterize the pressure on the drum by the concrete flow at different time during the rotation process of the drum of the concrete mixer truck during transportation. Preferably, the pressure sensor is arranged at the bottom of the drum and penetrates the drum, with the connected rod immersed in concrete. Preferably, the rheological shear rod is a sheet-like rod, such as a square-like rod, with a rectangular cross-section. By adjusting the length and cross-sectional shape of the sheet-like rod according to the resistance experienced by shear flow of different concretes, a more balanced resistance value can be obtained, further improving the accuracy of workload detection. In other embodiments, the rheological shear rod may also be other rod-shaped shear components, such as circular-like rods, elliptical-like rods, etc. The designed rheological shear device can be disassembled, cleaned, and worn parts of the designed rheological shear device can be replaced. In the embodiments of the present disclosure, the rheological shear device can also be installed at the middle position of the rotated drum of the concrete mixer truck and penetrate the drum.

In S2, based on the pressure signal corresponding to different time during the rotation of the drum of the concrete mixer truck, the relationship between the rotation speed of the drum of the concrete mixer truck and the resistance experienced by the rheological shear rod is obtained. The rheological shear rod inserted into the concrete shears the concrete during rotating with the drum, and different rotation speeds correspond to different shear resistances. In the rheological sense, the rotation speed of the drum is proportional to the shear rate, and the resistance experienced by the rheological shear rod is proportional to the shear stress. Therefore, the relationship between the rotation speed of the drum of the concrete mixer truck and the resistance experienced by the rheological shear rod can characterize the relationship between the shear speed of concrete and shear resistance of concrete. There is a linear relationship between shear speed and shear resistance. Therefore, the intercept corresponding to the relationship between the rotation speed of the drum of the concrete mixer truck and the resistance experienced by the rheological shear rod represents the structural strength (i.e. yield value) of the ready-mix concrete coarse suspension. A zero intercept represents that the particles of the concrete coarse suspension are in a fully dispersed state, and the slope of the straight line represents the plastic viscosity. The yield value and plastic viscosity are used as rheological parameters to reflect the rheological properties of concrete. If the relationship between the rotation speed of the drum of the concrete mixer truck and the resistance experienced by the rheological shear rod obtained over a period of time has deviated from the linear relationship, it can be concluded to some extent that the current rheological properties of the concrete have undergone significant changes. The changes in rheological properties over time are used to characterize the changes in concrete workability over time. In the embodiments of the present disclosure, workability includes slump, expansion, and the duration of slump to stability. The slump and expansion of concrete under self weight mainly depend on the yield value of ready-mix concrete, and the duration of slump to stability mainly depends on the plastic viscosity.

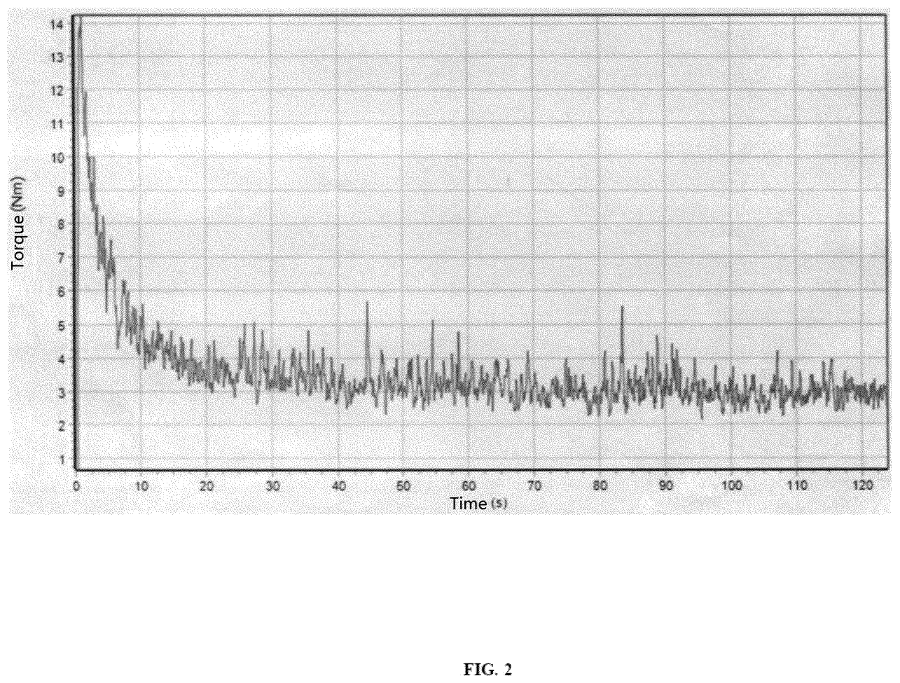

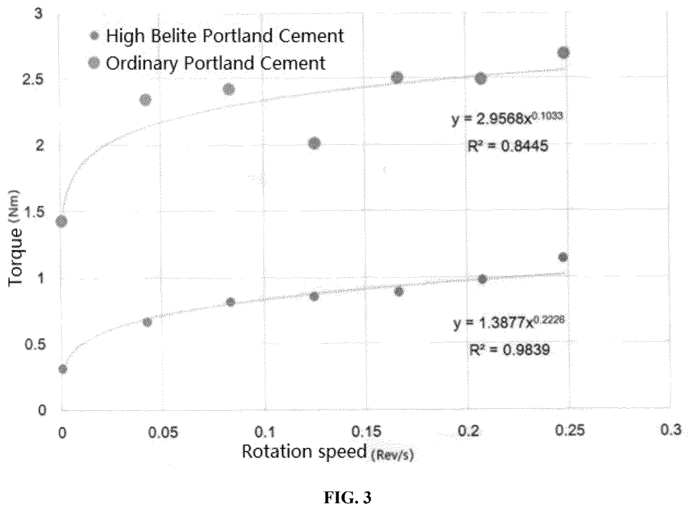

As a preferred implementation method, the pressure signal of the pressure sensor is transmitted to the remote monitoring center through a wireless signal transmitter corresponding to the pressure sensor, achieving real-time remote monitoring of changes in workability of concrete during transportation. In the embodiments of the present disclosure, the wireless signal transmitter is a 4G (the 4th generation mobile communication technology) wireless signal transmitter, which is installed outside the drum of the concrete mixer truck. In other embodiments, the wireless signal transmitter may also be other wireless signal transmitters adapted to the pressure signal of the pressure sensor. The remote monitoring center obtains the relationship among rotation speed of the drum, rod resistance, and transportation time based on the received relationship data of rotational speed (rotation speed) of the drum, pressure, and time, and then obtains the changes in rheological parameters over time during transportation. In addition, corresponding stress data can be obtained according to the pressure data of the sensor, and the law of changes in resistance over time can be obtained according to the rotation speed of the drum and the corresponding stress. The data reflecting changes in pressure, stress, apparent viscosity, and temperature over time can be served as one of the basis for regulating the workability of concrete in the future. As shown in FIG. 2, it is a schematic diagram of the law of changes in the rod torque in the concrete over the test time at a certain time point. The stable value can represent the apparent viscosity at this time. FIG. 3 shows the relationship between the rod torque and rotation speed, representing the schematic diagram of concrete rheological test. R² in FIG. 3 is the set parameter.

Furthermore, based on the rheological shear of concrete at different time during transportation, the apparent viscosity corresponding to a certain rotation speed, i.e. flow resistance, can also be calculated. In the embodiments of the present disclosure, the yield value and plastic viscosity before transportation are obtained based on the relationship between the resistance of the drum of the tested concrete mixer truck at multiple rotation speeds. During transportation, the apparent viscosity of concrete at normal rotation speeds of the drum can be characterized by the magnitude of the “resistance/rotation speed” value. The change in apparent viscosity over time can also reflect the change in concrete workability over time.

As a further design of the present disclosure, the present disclosure has considered that the hydration of cement in concrete leads to a decrease in moisture, an increase in solid phase, and an increase in viscosity. The temperature increase caused by hydration accelerates the hydrated reaction and also affects the changes in concrete workability over time. Therefore, the method for detecting workability of concrete in concrete mixer truck provided in the present disclosure further includes following steps.

A real-time measurement of temperature T during concrete transportation is performed to construct the relationships between yield value, plastic viscosity, apparent viscosity, and temperature T over time. The temperature T includes the temperature of concrete inside the drum of the concrete mixer truck and/or temperature of the environment inside the drum of the concrete mixer truck and temperature of the environment outside the drum of the concrete mixer truck, to obtain the difference between the temperature inside the drum of the concrete mixer truck and the temperature outside the drum of the concrete mixer truck.

In practical applications, an adaptive selection can be made according to the climate environment in the south and north. For example, in summer in the south and winter in the north, there is a large temperature difference between the inside and outside of the drum of the concrete mixer truck. In these cases, the data of the temperature difference between the inside and outside can be selected to construct the relationships between yield value, plastic viscosity, apparent viscosity, and temperature T over time.

In the embodiments of the present disclosure, a concrete temperature sensor is used to measure the temperature T during concrete transportation in real time. During measuring the temperature inside the drum of the concrete mixer truck, the concrete temperature sensor faces the inside of the drum and is in contact with the concrete inside the drum. The transmitter of the temperature sensor signal is arranged outside the drum and transmits the corresponding temperature signal wirelessly.

It should be noted that the rheological properties and temperature test of concrete is only related to the properties of the concrete inside the drum, and are not related to the shape of the drum or the loading amount of concrete inside the drum.

The data of the rheological shear rod when the rheological shear rod leaves the concrete with the rotation of the drum is determined and eliminated by the calculation software.

In the method for detecting workability of concrete in concrete mixer truck according to the present disclosure, a rheological shear device arranged inside the drum of the concrete mixer truck is used to directly convert the resistance experienced by the rheological shear device when concrete flows at different time during the rotation process of the drum of the concrete mixer truck into a pressure signal. Based on the pressure signal corresponding to different time during the rotation process of the drum of the concrete mixer truck, the relationship between the rotation speed of the drum of the concrete mixer truck and the resistance experienced by the rheological shear rod can be obtained. The rheological shear rod inserted into the concrete serves as a shear rod, which shears the concrete when rotates with the drum. Different rotation speeds correspond to different shear resistances. In the rheological sense, the rotation speed of the drum is proportional to the shear rate. Therefore, the obtained relationship between the rotation speed of the drum of the concrete mixer truck and the resistance experienced by the rheological shear rod can characterize the relationship between the shear speed and shear resistance of concrete. There is a linear relationship between shear speed and shear resistance, so the intercept corresponding to the relationship between the rotation speed of the drum of the concrete mixer truck and the resistance experienced by the rheological shear rod represents the yield value, and the slope of the straight line represents the plastic viscosity. The apparent viscosity at a certain rotation speed can be calculated based on the yield value and plastic viscosity at different time during transportation. The changes in yield value, plastic viscosity, and apparent viscosity over time can reflect the changes in the concrete workability over time. Therefore, the present disclosure achieves direct detection of the workability of fresh concrete, with the advantages of high accuracy and strong real-time performance.

Furthermore, the present disclosure takes into account that the hydration of cement in concrete leads to a decrease in moisture, an increase in solid phase, and an increase in viscosity. The temperature increase caused by hydration accelerates the hydrated reaction and also affects changes in the concrete workability over time. Therefore, the method of the present disclosure further includes a real-time measurement of the temperature T during concrete transportation to construct the relationships between the yield value, the plastic viscosity, the apparent viscosity, and the temperature T over time, further improving the accuracy of direct measurement of the workability of fresh concrete.

### Embodiment 2

On the basis of the Embodiment 1, a method for regulating workability during concrete transportation is provided in the Embodiment 2. The method in the Embodiment 2 includes following steps.

A database of the relationship between rheological properties and workability of concrete with different categories and strength grades under different transportation conditions over time is constructed. The values in the database are used as standard values for comparison with subsequent real-time measurement values. Through this database, the relationship between the rheological properties of each concrete product (different strength grades of concrete) and different transportation conditions (transportation by concrete mixer truck, transportation time, environmental temperature) over time can be obtained. Due to the different properties of engineering concrete categories and the inability to unify transportation conditions, it is necessary to continuously optimize and accurately determine the relevant relationship based on big data. In the embodiments of the present disclosure, the database not only includes the apparent viscosity data of a certain rotation speed, and the yield value and the plastic viscosity of a certain concrete under different rotation speeds of agitating during transportation, but also includes the data reflecting changes in pressure measured by the original pressure sensor over time, as well as the data reflecting changes in concrete temperature under transportation conditions over time.

After the fresh concrete produced in the factory is unloaded into the drum of the concrete-transport mixer truck, samples are extracted according to quality management regulations for concrete workability test, and the initial value of concrete workability can be obtained. At the set rotation speed of the drum, the rheological properties of the concrete before transportation are measured using the detection method described in the Embodiment 1, and the initial values of the rheological properties can be obtained. The test data is compared with the relevant concrete standard value data in the database to confirm the initial workability and initial rheological properties of the concrete before leaving the factory. When the initial workability meets the requirements, the concrete-transport mixer truck will leave the factory.

After the concrete-transport mixer truck leaves the factory, during transportation, the workability detecting method in the Embodiment 1 is used to continuously detect the apparent viscosity of the concrete at different time. If necessary, the apparent viscosity is regulated by adding admixtures until the concrete-transport mixer truck reaches the delivery location. Before delivery and unloading, the rheological properties of the concrete can be tested again at the same rotation speed as before leaving the factory to confirm that the workability of the concrete meets the requirements. In the embodiments of the present disclosure, the rheological shear resistance of concrete is tested once every rotation of the drum of the concrete mixer truck. In other embodiments, the testing time interval can also be adjusted according to actual needs.

As a specific implementation method, the method described in the Embodiment 1 can be used to test the relationship between rotation speed and shear resistance at several lower rotation speeds after loading the concrete and before the transportation, in order to construct a time-varying relationship database of the apparent viscosity. During transportation, the concrete mixer truck usually agitate concrete at a lower speed. During the process, the rheological properties of the concrete are detected at regular intervals (e.g. 3 minutes, this parameter can be adjusted), and the apparent viscosity at this rotation speed is represented by the shear resistance at this rotation speed. The apparent viscosity tested and calculated during transportation is compared with the apparent viscosity value at the initial transportation to calculate a difference to determine the trend of the difference over time during the current period. According to the relationship between workability and apparent viscosity, if the trend of the difference over time during the current period leads to a decrease in workability (an increase in the apparent viscosity of concrete) and reaches the preset warning value, the program control will be triggered to automatically supplement admixtures to regulate the workability of the concrete to meet the required requirements of the workability. During this period, if only a portion of the apparent viscosity differences reflects a decrease in workability, an optimization algorithm can be used based on the corresponding yield value, plastic viscosity, and raw data (including the aforementioned data of pressure, stress, shear resistance, and temperature over time) to intelligently determine whether automatic addition of admixtures is needed. The optimization algorithm can be an intelligent-agent optimization algorithm or an intelligent optimization algorithm such as a neural network.

In addition, the amount of supplementing admixtures and duration of supplementation can be automatically optimized and decided according to the set target of concrete workability at the time of delivery by using an optimization algorithm, thereby ensuring that the workability of concrete meets the requirements when the transport vehicle arrives at the delivery location.

In the embodiments of the present disclosure, a device for automatically supplementing spray admixtures is used for automatically supplement admixtures. The device for automatically supplementing spray admixtures includes: an admixture liquid storage tank carried and fixed outside the drum of the concrete mixer truck, an automatic-control spraying system installed at the discharge port of the drum of the concrete mixer truck, and an admixture-flow metering system. During transportation, the rheological properties and temperature of the concrete inside the drum of the concrete mixer truck are continuously measured. By comparing the measured rheological properties with the required rheological properties, the device for automatically supplementing spray admixtures is used to automatically supplement admixtures to regulate the rheological properties of the concrete within the set range.

Preferably, the Embodiment 2 also includes constructing the relationship among the amount of supplementing admixtures and different categories of the admixtures and workability of concrete with strength grades and apparent viscosity changes, as well as the relationship between the increase of concrete water cement ratio due to the supplementation of water-based admixtures and the influence on concrete strength. Based on the constructed relationships, without affecting the concrete strength to meet the design requirements, the quantity and frequency of each spray of supplementing admixtures are regulated to ensure that the apparent viscosity (workability) of the concrete meets the requirements.

Preferably, the addition of admixtures during transportation can be used as the basis for designing and optimizing the mix proportion of concrete. According to the similar concrete and the similar environmental temperatures in big data, as well as and the estimation of the duration of transportation, the dosage of admixtures can be optimized to avoid excessively increasing the initial dosage of the admixtures, achieving the goal of meeting the workability requirements of concrete and reducing the cost of admixtures.

As a preferred implementation method, the Embodiment 2 also includes judging the trend of changes in concrete temperature over time. If the trend of changes in concrete temperature over time deteriorates the workability and reaches the preset warning value, the program control can be triggered to automatically supplement the admixtures to regulate the workability of the concrete. If the temperature rise of concrete is not high and the rheological properties do not change significantly over time in the direction of increasing viscosity, or do not reach the warning value, then admixtures may not be added.

Preferably, the categories of admixtures added during transportation are generally water-based high-efficiency water reducers, retarders, retarding water reducers, and other functional admixtures. For example, in winter construction, early strength antifreezing agents can be added before concrete unloading.

Preferably, for high-performance concrete and key-engineering concrete, owners and project supervisors can monitor the quality management and workability of concrete in real time through online shared information.

The workability and changes thereof during concrete production and transportation can be monitored and regulated, which can be an effective measure for factory quality management. The process includes following steps.

The central control room (or remote monitoring center) of the concrete factory can receive signals wirelessly and monitor the time and actual route of the concrete transport vehicle during transportation online. It can grasp the possible unexpected overtime situations that may occur during concrete transportation, the possible unexpected overtime situation include traffic congestion during transportation and long waiting time for delivery and unloading at the construction site. Different regulation measures can be taken according to the location and dynamics of the transport vehicle, such as a slow rotation during transportation, a slow or accelerated agitation during static waiting, and a rapid agitation and homogenization before unloading. It can promptly handle problems reported by drivers or on-site technicians.

Whether regulatory intervention is necessary can be determined according to real-time monitoring of changes in concrete apparent viscosity (or rheological properties data such as yield values and plastic viscosity at different time) and temperature. In the case where the quality requirements of the contract are still not met after taking regulatory intervention measures, timely decisions should be made on the disposal measures of the concrete in the concrete mixer truck, such as converting the concrete to other uses or transporting the concrete back to waste, in order to reduce economic losses and prevent concrete engineering quality accidents.

In the method for regulating workability of concrete during concrete transportation according to the present disclosure, according to workability requirements and changing workability, admixtures can be automatically and quantitatively sprayed into the concrete in the concrete mixer truck according to program settings under software control, achieving timely micro regulation or pre-regulation of the workability of concrete during transportation, ensuring that the workability of concrete meets design requirements. The present disclosure can more accurately control the changes in concrete workability during transportation, further optimize the design and adjustment of mix proportion of the concrete, improve the quality level of concrete engineering, and solve the problem of quality monitoring and regulation deficiencies in concrete transportation.

### Embodiment 3

A system for detecting workability of concrete during transportation is provided by the Embodiment 3 according to the present disclosure. The system includes: a rheological shear device arranged inside a drum of a concrete mixer truck, a rheological parameter calculation module, and a workability real-time detection module.

The rheological shear device arranged inside a drum of a concrete mixer truck is configured to directly convert resistance experienced by the rheological shear device when concrete flows at different time during rotation of the drum into a pressure signal. The rheological shear device includes a rheological shear rod and a pressure sensor. The rheological shear rod inserts into the concrete inside the drum and is used to directly detect a resistance experienced when concrete flows during the rotation of the drum, and the resistance is converted into a pressure experienced by the pressure sensor through a law of lever.

The rheological parameter calculation module is configured to obtain a relationship between a rotation speed of the drum and a resistance experienced by the rheological shear rod at different time based on the pressure signal. An intercept of a straight line of the relationship represents a yield value, and a slope of the straight line of the relationship represents a plastic viscosity.

The workability real-time detection module is configured to reflect a workability by using the yield value and the plastic viscosity, and obtain a time-varying relationship of the workability during concrete transportation, thereby achieving a real-time detection of the workability.

In some embodiments, the system also includes a temperature sensor.

The temperature sensor is configured to measure temperature T during concrete transportation in real time to construct relationships between the yield value, the plastic viscosity, an apparent viscosity, and the temperature T over time. The temperature T includes temperature of the concrete inside the drum of the concrete mixer truck, and/or ambient temperature inside the drum of the concrete mixer truck and ambient temperature outside the drum of the concrete mixer truck to obtain a difference between the temperature inside the drum of the concrete mixer truck and the temperature outside the drum of the concrete mixer truck.

The specific implementation methods of each module, device, or component mentioned above can be found in the corresponding description in the Embodiment 1, and will not be repeated here.

### Embodiment 4

A system for regulating workability during concrete transportation is provided in the Embodiment 4 according to the present disclosure. The system includes: a database construction module, a workability detection system, a calculation module, and a device for automatically supplementing spray admixtures.

The database construction module is configured to pre-construct a time-varying relationship database of apparent viscosity of concrete with different categories, strength grades, and workability requirements under different transportation conditions; and is configured to construct a database reflecting a relevant relationship between the workability of concrete and the apparent viscosity.

The workability detection system is configured to detect the workability of concrete at different time during transportation in real time until the concrete mixer truck reaches a delivery location. The workability detection system is the system for detecting workability during concrete transportation according to the Embodiment 3.

The calculation module is configured to calculate a corresponding apparent viscosity at a regular interval during transportation based on real-time detected yield value and plastic viscosity, and compare the apparent viscosity with an apparent viscosity in the database to obtain a trend of apparent viscosity differences over time during this period.

The device for automatically supplementing spray admixtures is configured to automatically supplement in real time admixtures when the trend of apparent viscosity differences over time during this period leads to a decrease in workability and when the trend reaches a preset warning value, to regulate the workability of concrete in real time.

The specific implementation methods or structures of each module or device can be found in the description in the corresponding embodiments, and will not be repeated here.

The present disclosure directly tests the rheological properties of ready-mix concrete of the concrete mixer truck during transportation, and determines, monitors, predicts, and regulates the workability and changes thereof through the relationship between the rheological properties and workability, as well as the influence of environmental temperature and cement hydration on the relationship, in order to meet the construction requirements for concrete workability and strength performance. As long as the various changes in the rheological properties of concrete are within the spirit and scope of the present disclosure defined and determined by the protected technical solution, and these changes with cement hydration, environment, and time are obvious, all inventions and creations utilizing the concept of the present disclosure are protected.

It is easy for those skilled in the art to understand that aforementioned descriptions are only the preferred embodiments of the present disclosure and are not intended to limit the present disclosure. Any modifications, equivalent substitutions, and improvements made within the spirit and principles of the present disclosure should be included within the scope of protection of the present disclosure.

What is claimed is:

1. A method for detecting workability of concrete in a concrete mixer truck, comprising:
directly converting resistance experienced by a rheological shear rod exposed to concrete flowing during rotation of a drum of a concrete mixer truck into a pressure signal by using a rheological shear device arranged inside the drum; wherein the rheological shear device comprises the rheological shear rod and a pressure sensor; wherein the rheological shear rod inserts into concrete inside the drum and is used to directly detect a resistance experienced by the concrete during the rotation of the drum when the concrete is shear flowed, and the resistance is converted into a pressure experienced by the pressure sensor through a law of lever; wherein the pressure experienced by the pressure sensor is used to characterize pressures exerted on the drum when the concrete flows at different times during the rotation of the drum of the concrete mixer truck during transportation; wherein the pressure sensor is arranged on bottom of the drum and penetrates the drum; wherein the rheological shear rod is immersed into the concrete; and wherein the rheological shear rod is a sheet-like rod, and a length and a cross-sectional shape of the sheet-like rod are adjusted according to resistance experienced by the sheet-like rod during shear flow of different concretes;
based on the pressure signal, obtaining a relationship between a rotation speed of the drum and a resistance experienced by the rheological shear rod at different time; wherein an intercept of a straight line of the relationship represents a yield value, and a slope of the straight line of the relationship represents a plastic viscosity; and
reflecting a workability by using the yield value and the plastic viscosity, and obtaining a time-varying relationship of the workability during concrete transportation, thereby achieving a real-time detection of the workability.

2. The method for detecting workability of concrete in a concrete mixer truck according to claim 1, wherein the method further comprises: measuring temperature T during the concrete transportation in real time to construct relationships between the yield value, the plastic viscosity, an apparent viscosity, and the temperature T over time; wherein the temperature T comprises temperature of the concrete inside the drum of the concrete mixer truck, and wherein the temperature T optionally further comprises an ambient temperature inside the drum of the concrete mixer truck and an ambient temperature outside the drum of the concrete mixer truck to obtain a difference between the ambient temperature inside the drum of the concrete mixer truck and the ambient temperature outside the drum of the concrete mixer truck.

3. The method for regulating workability of concrete in a concrete mixer truck, comprising: pre-constructing a database of a time-varying relationship of apparent viscosity of concrete with different categories, strength grades, and workability requirements under different transportation conditions;
detecting the workability of concrete at different times during transportation in real time by using the method for detecting workability of concrete in concrete mixer truck according to claim 1 until the concrete mixer truck reaches a delivery location;
during transportation, based on real-time detected yield value and plastic viscosity, calculating a corresponding apparent viscosity at a time period; comparing the apparent viscosity with an apparent viscosity in the database to obtain a trend of apparent viscosity difference over time during the time period; and
automatically supplementing admixtures when the trend of apparent viscosity difference over time during the time period leads to a decrease in workability and when the trend reaches a preset warning value, to regulate the workability of concrete in real time.

4. The method for regulating workability of concrete in a concrete mixer truck, comprising: pre-constructing a database of a time-varying relationship of apparent viscosity of concrete with different categories, strength grades, and workability requirements under different transportation conditions;
detecting the workability of concrete at different time during transportation in real time by using the method for detecting workability of concrete in concrete mixer truck according to claim 2 until the concrete mixer truck reaches a delivery location;
during transportation, based on real-time detected yield value and plastic viscosity, calculating a corresponding apparent viscosity at a time period; comparing the apparent viscosity with an apparent viscosity in the database to obtain a trend of apparent viscosity difference over time during the time period; and
automatically supplementing admixtures when the trend of apparent viscosity difference over time during the time period leads to a decrease in workability and when the trend reaches a preset warning value, thus regulating the workability of concrete in real time.

5. The method for regulating workability of concrete in a concrete mixer truck according to claim 3, wherein during the time period, if workability reflected by apparent viscosity differences decreases, the method for regulating workability further comprises:
based on the apparent viscosity differences, as well as a yield value, a plastic viscosity, and pressure data measured by the pressure sensor corresponding to the apparent viscosity differences, deciding, by using an optimization algorithm, whether the admixtures are automatically supplemented.

6. The method for regulating workability of concrete in a concrete mixer truck according to claim 4, wherein during the time period, if workability reflected by apparent viscosity differences decreases, the method for regulating workability further comprises:
based on the apparent viscosity differences, as well as a yield value, a plastic viscosity, and pressure data measured by the pressure sensor corresponding to the apparent viscosity differences, deciding, by using an optimization algorithm, whether the admixtures are automatically supplemented.

7. The method for regulating workability of concrete in a concrete mixer truck according to claim 3, wherein a variety of the admixtures, an amount of each supplement, and frequency of supplementation of the admixtures are automatically optimized and determined using an optimization algorithm according to a set target of the workability of concrete when delivery.

8. The method for regulating workability of concrete in a concrete mixer truck according to claim 4, wherein a variety of the admixtures, an amount of each supplement, and frequency of supplement of the admixtures are automatically optimized and determined using an optimization algorithm according to a set target of the workability of concrete when delivery.

9. The method for regulating workability of concrete in a concrete mixer truck according to claim 5, wherein a variety of the admixtures, an amount of each supplement, and frequency of supplement of the admixtures are automatically optimized and determined using an optimization algorithm according to a set target of the workability of concrete when delivery.

10. The method for regulating workability of concrete in a concrete mixer truck according to claim 6, wherein a variety of the admixtures, an amount of each supplement, and frequency of supplement of the admixtures are automatically optimized and determined using an optimization algorithm according to a set target of the workability of concrete when delivery.

11. The method for regulating workability of concrete in a concrete mixer truck according to claim 7, further comprises: monitoring time and an actual route of a concrete mixer truck during transportation and an unloading and delivery location and time in real time, to assist in regulation of the workability of concrete.

12. The method for regulating workability of concrete in a concrete mixer truck according to claim 8, further comprises: monitoring time and an actual route of a concrete mixer truck during transportation and an unloading and delivery location and time in real time, to assist in regulation of the workability of concrete.

13. The method for regulating workability of concrete in a concrete mixer truck according to claim 9, further comprises: monitoring time and an actual route of a concrete mixer truck during transportation and an unloading and delivery location and time in real time, to assist in regulation of the workability of concrete.

14. The method for regulating workability of concrete in a concrete mixer truck according to claim 10, further comprises: monitoring time and an actual route of a concrete mixer truck during transportation and an unloading and delivery location and time in real time, to assist in regulation of the workability of concrete.

15. A system for detecting workability of concrete in a concrete mixer truck, comprising:
a rheological shear device arranged inside a drum of a concrete mixer truck, configured to directly convert resistance experienced by a rheological shear rod exposed to concrete flowing during rotation of the drum into a pressure signal; wherein the rheological shear device comprises the rheological shear rod and a pressure sensor; wherein the rheological shear rod inserts into concrete inside the drum and is used to directly detect a resistance experienced by the concrete during the rotation of the drum when the concrete is shear flowed, and the resistance is converted into a pressure experienced by the pressure sensor through a law of lever; wherein the pressure experienced by the pressure sensor is used to characterize a pressure exerted on the drum when the concrete flows at different times during the rotation of the drum of the concrete mixer truck during transportation; wherein the pressure sensor is arranged on bottom of the drum and penetrates the drum; wherein the rheological shear rod is immersed into the concrete; and wherein the rheological shear rod is a sheet-like rod, and a length and a cross-sectional shape of the sheet-like rod are adjusted according to resistance experienced by the sheet-like rod during shear flow of different concretes;
a rheological parameter calculation module, configured to obtain a relationship between a rotation speed of the drum and a resistance experienced by the rheological shear rod at different times based on the pressure signal; wherein an intercept of a straight line of the relationship represents a yield value, and a slope of the straight line of the relationship represents a plastic viscosity; and
a workability real-time detection module, configured to reflect a workability by using the yield value and the plastic viscosity, and obtain a time-varying relationship of the workability during concrete transportation, thereby achieving a real-time detection of the workability.

16. The system for detecting workability of concrete in a concrete mixer truck according to claim 15, further comprising: a temperature sensor;
wherein the temperature sensor is configured to measure temperature T during the concrete transportation in real time to construct relationships between the yield value, the plastic viscosity, an apparent viscosity, and the temperature T over time; wherein the temperature T comprises temperature of the concrete inside the drum of the concrete mixer truck, and wherein the temperature T optionally further comprises an ambient temperature inside the drum of the concrete mixer truck and an ambient temperature outside the drum of the concrete mixer truck to obtain a difference between the ambient temperature inside the drum of the concrete mixer truck and the ambient temperature outside the drum of the concrete mixer truck.

17. A system for regulating workability of concrete in a concrete mixer truck, comprising:
a database construction module, configured to pre-construct a database of a time-varying relationship of apparent viscosity of concrete with different categories, strength grades, and workability requirements under different transportation conditions;
a workability detection system, configured to detect the workability of concrete at different time during transportation in real time until the concrete mixer truck reaches a delivery location; wherein the workability detection system is the system for detecting workability of concrete in concrete mixer truck according to claim 15;
a calculation module, configured to calculate a corresponding apparent viscosity at a time period during transportation based on real-time detected yield value and plastic viscosity; and
compare the apparent viscosity with an apparent viscosity in the database to obtain a trend of apparent viscosity difference over time during the time period; and
a device for automatically supplementing spray admixtures, configured to automatically supplement admixtures when the trend of apparent viscosity difference over time during the time period leads to a decrease in workability and when the trend reaches a preset warning value, thus regulating the workability of concrete in real time.

18. A system for regulating workability of concrete in a concrete mixer truck, comprising:
a database construction module, configured to pre-construct a database of a time-varying relationship of apparent viscosity of concrete with different categories, strength grades, and workability requirements under different transportation conditions;
a workability detection system, configured to detect the workability of concrete at different time during transportation in real time until the concrete mixer truck reaches a delivery location; wherein the workability detection system is the system for detecting workability of concrete in concrete mixer truck according to claim 16;
a calculation module, configured to calculate a corresponding apparent viscosity at a time period during transportation based on real-time detected yield value and plastic viscosity; and compare the apparent viscosity with an apparent viscosity in the database to obtain a trend of apparent viscosity difference over time during the time period; and
a device for automatically supplementing spray admixtures, configured to automatically supplement admixtures when the trend of apparent viscosity difference over time during the time period leads to a decrease in workability and when the trend reaches a preset warning value, thus regulating the workability of concrete in real time.
